(19) United States
(12) Patent Application Publication
(10) Pub. No.: US 2014/0343380 A1
(43) Pub. Date: Nov. 20, 2014

(54) Correlating Sensor Data Obtained from a Wearable Sensor Device with Data Obtained from a Smart Phone

(71) Applicants: Abraham Carter, Salt Lake City, UT (US); David Scott, North Salt Lake City, UT (US)

(72) Inventors: Abraham Carter, Salt Lake City, UT (US); David Scott, North Salt Lake City, UT (US)

(21) Appl. No.: 14/279,140

(22) Filed: May 15, 2014

Related U.S. Application Data
(60) Provisional application No. 61/823,830, filed on May 15, 2013.

(51) Int. Cl. A61B 5/00; A61B 5/024; A61B 5/145; A61B 5/11

(52) U.S. Cl. CPC A61B 5/7246; A61B 5/6802; A61B 5/11; A61B 5/4806; A61B 5/024; A61B 5/14546; A61B 5/0002; A61B 5/6898
USPC 600/309; 600/300; 600/595; 600/508

(57) ABSTRACT

Sensor data obtained from a wearable sensor device can be correlated with sensor data obtained from a smart phone or other mobile device. The correlation of the data from the two sources can enable the determination of why a person performs some action during sleep. In a particular example, motion data obtained from a wearable sensor device can be correlated with audio or visual data obtained by a sensor on a smart phone. In this way, it can be determined whether a person moved in response to a sound or light perceived during sleep. Additionally, the correlation of the data from the two sources can also provide additional information about how a user performs an activity such as exercise.

### CROSS-REFERENCE TO RELATED APPLICATIONS

This application claims priority to U.S. Provisional Patent Application No. 61/823,830 which was filed on May 15, 2013.

### BACKGROUND

Many wearable sensor devices exist for tracking various metrics of a person wearing the device. For example, some wearable sensor devices include accelerometers and/or gyroscopes which can detect when the wearable sensor device moves. Such devices can be used to track when a person moves or the amount of movement the person makes while wearing the device. Other wearable sensor devices can include biometric sensors such as a pulse oximeter for measuring a person's hemoglobin saturation, a heart rate monitor, a thermometer, etc.

In some cases, wearable sensor devices are used to monitor a person's sleep patterns. For example, a wearable sensor device with an accelerometer or gyroscope can be worn by a person at night to track when the person moves thereby giving an indication of how often the person tosses and turns during sleep. Also, some applications for smart phones allow the smart phone to be used as a wearable sensor device to track the person's movement during sleep. These applications can also provide functionality for recording audio during sleep to detect when a person snores, talks, or struggles to breath.

Accordingly, current wearable sensor devices (including smart phones configured to function as wearable sensor devices) can provide a substantial amount of information regarding actions a person makes during sleep. By using such devices, a person can be informed of when he moves, snores, talks, etc. during sleep. However, the information provided by such devices does not provide any information regarding why the person may have moved, snored, talked, etc. during sleep.

In other cases, wearable sensor devices (or smart phone configured to be used as wearable sensor devices) can be used to track various parameters during a user's activity such as exercise. Such devices can also provide a substantial amount of information regarding the user's activity. However, the information provided by the sensors of the wearable sensor devices is not correlated with any sensor data obtained using one or more sensors of a mobile device which receives the sensor data from the wearable sensor devices.

### BRIEF SUMMARY

The present invention extends to methods, systems, and computer program products for correlating sensor data obtained from a wearable sensor device with sensor data obtained from a smart phone. The correlation of the data from the two sources can enable the determination of why a person performs some action during sleep. In a particular example, motion data obtained from a wearable sensor device can be correlated with audio or visual data obtained by a sensor on a smart phone. In this way, it can be determined whether a person moved in response to a sound or light perceived during sleep. Additionally, the correlation of the data from the two sources can also provide additional information about how a user performs an activity such as exercise.

In one embodiment, the present invention is implemented as a method, performed by a mobile device, for correlating sensor data received from a wearable sensor device with sensor data received from one or more sensors within the mobile device. First sensor data is received from one or more sensors of the mobile device. The first sensor data represents an environmental occurrence while a user is sleeping. The first sensor data is stored with an indication of a first time at which the first sensor data was generated. Second sensor data is received from one or more wearable sensor devices that are worn by the user while the user is sleeping. The second sensor data is generated by one or more sensors in the one or more wearable sensor devices. The second sensor data represents a movement the user made while sleeping. The second sensor data is stored with an indication of a second time at which the second sensor data was generated. The first and second sensor data is analyzed including determining that the duration of time between the first time and the second time is below a specified threshold. A correlation is created between the environment occurrence and the movement.

The environmental occurrence may be a sound or a light. In some instances, the correlation may include a strength that is based on one or more of an intensity of the environmental occurrence, or the duration of time between the first time and the second time.

In another embodiment, the present invention is implemented as a method, performed by a mobile device, for correlating sensor data received from a wearable sensor device with sensor data received from one or more sensors within the mobile device. First sensor data is received from one or more sensors of the mobile device. The first sensor data is stored with an indication of a first time at which the first sensor data was generated. Second sensor data is received from one or more wearable sensor devices that are worn by the user while the user is sleeping. The second sensor data is generated by one or more sensors in the one or more wearable sensor devices. The second sensor data represents a change in a physiological parameter of the user while the user is sleeping. The second sensor data is stored with an indication of a second time at which the second sensor data was generated. The first and second sensor data is analyzed to determine that the duration of time between the first time and the second time is below a specified threshold. A correlation is created between the environment occurrence and the change in the physiological parameter.

The physiological parameter may be the user's heart rate or hemoglobin saturation among other parameters. In some instances the correlation may include a strength that is based on one or more of an intensity of the environmental occurrence, or the duration of time between the first time and the second time.

In another embodiment, the present invention is implemented as one or more computer storage media storing computer executable instructions which when executed implement a method for correlating sensor data received from a wearable sensor device with sensor data received from one or more sensors within a mobile device. First sensor data is received from one or more sensors of the mobile device. The first sensor data is stored with an indication of a first time at which the first sensor data was generated. Second sensor data is received from one or more wearable sensor devices that are worn by the user. The second sensor data is generated by one or more sensors in the one or more wearable sensor devices. The second sensor data is stored with an indication of a second time at which the second sensor data was generated. The first and second sensor data is analyzed to determine that the duration of time between the first time and the second time is below a specified threshold. A correlation is created between first sensor data and the second sensor data based on the duration of time being below the specified threshold.

The first sensor data may represent an audible or visible occurrence while the second sensor data may represent motion. The second sensor data may be generated while the user is sleeping or exercising. When the second sensor data represents motion, the correlation can indicate that the motion likely occurred as a result of an occurrence represented by the first sensor data. In some instances, an indication of the correlation may be displayed on the mobile device.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates an exemplary computing environment in which the present invention can be implemented;

FIGS. 2A-2C illustrate how motion data generated by a wearable sensor device can be correlated with audible data generated by a microphone of a mobile device; and

FIG. 3 illustrates how sensor data generated by a wearable sensor device while performing an activity can be correlated with sensor data generated by one or more sensors of a mobile device used to receive the sensor data generated by the wearable sensor device.

### DETAILED DESCRIPTION

The present invention extends to methods, systems, and computer program products for correlating sensor data obtained from a wearable sensor device with sensor data obtained from a smart phone. The correlation of the data from the two sources can enable the determination of why a person performs some action during sleep. In a particular example, motion data obtained from a wearable sensor device can be correlated with audio or visual data obtained by a sensor on a smart phone. In this way, it can be determined whether a person moved in response to a sound or light perceived during sleep. Additionally, the correlation of the data from the two sources can also provide additional information about how a user performs an activity such as exercise.

Embodiments of the present invention may comprise or utilize special purpose or general-purpose computers including computer hardware, such as, for example, one or more processors and system memory, as discussed in greater detail below. Embodiments within the scope of the present invention also include physical and other computer-readable media for carrying or storing computer-executable instructions and/or data structures. Such computer-readable media can be any available media that can be accessed by a general purpose or special purpose computer system.

Computer-readable media is categorized into two disjoint categories: computer storage media and transmission media. Computer storage media (devices) include RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other similarly storage medium which can be used to store desired program code means in the form of computer-executable instructions or data structures and which can be accessed by a general purpose or special purpose computer. Transmission media include signals and carrier waves.

Computer-executable instructions comprise, for example, instructions and data which, when executed by a processor, cause a general purpose computer, special purpose computer, or special purpose processing device to perform a certain function or group of functions. The computer executable instructions may be, for example, binaries, intermediate format instructions such as assembly language or P-Code, or even source code.

Those skilled in the art will appreciate that the invention may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like.

The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices. An example of a distributed system environment is a cloud of networked servers or server resources. Accordingly, the present invention can be hosted in a cloud environment.

Correlating Sensor Data Obtained from a Wearable Sensor Device Worn During Sleeping with Sensor Data Obtained from Sensors of a Mobile Device

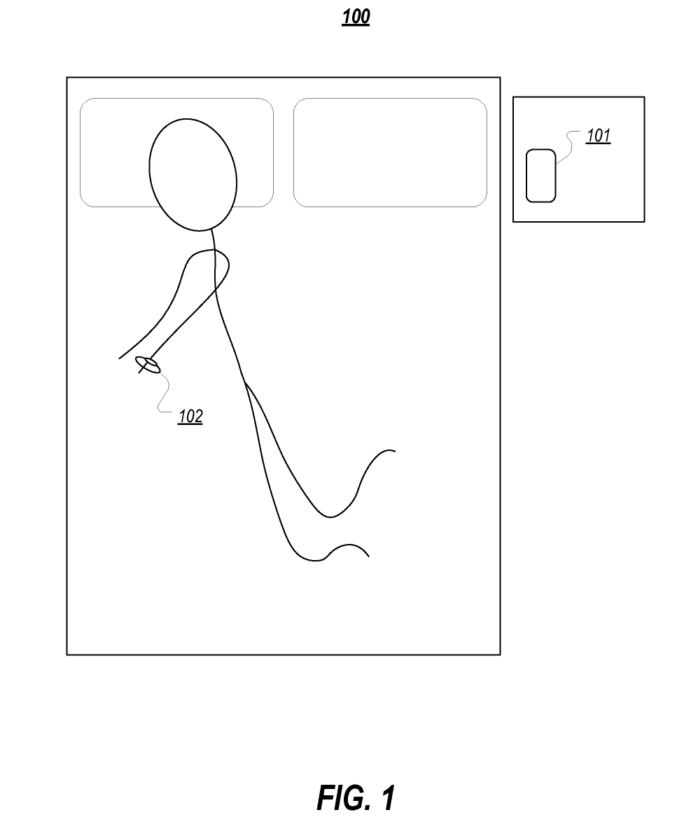

FIG. 1 illustrates an exemplary computer environment 100 in which the present invention can be implemented. Computer environment 100 includes a mobile device 101 and wearable sensor device 102 that is worn by a user during sleep. Mobile device 101 will typically be a user's smart phone; however, other mobile devices having sensors can also be used.

Wearable sensor device 102 can include one or more different types of sensors for detecting various parameters. In a particular embodiment, the sensors can include one or more of an accelerometer, a blood glucose sensor, a pulse oximeter, a skin temperature sensor, or a blood pressure sensor among others. Wearable sensor device 102 can include an interface for transmitting sensor data received from the one or more sensors of the wearable sensor device to mobile device 101.

An accelerometer in a wearable sensor device can be used to detect the movement of the user's body part on which the wearable sensor device is worn. For example, in the particular embodiment shown in FIG. 1, wearable sensor device 102 is worn around the user's wrist (e.g. as a bracelet) and includes an accelerometer for identifying when the user's arm moves. In such embodiments, wearable sensor device 102 can also include one or more sensors for detecting one or more of the user's physiological parameters such as heart rate, skin temperature, hemoglobin saturation, etc.

In some embodiments, more than one wearable sensor device can be worn. In such cases, each wearable sensor device can contain the same or different sensors. Each wearable sensor device can be configured to communicate directly with mobile device 101. Alternatively, one or more wearable sensor devices can be configured to communicate sensor data to another wearable sensor device which routes the sensor data to mobile device 101. Accordingly, the particular number of wearable sensor devices as well as the particular way that each wearable sensor device transmits sensor data to mobile device 101 is not essential to the invention.

In some embodiments, multiple wearable sensor devices can be used so that each individual wearable sensor device can be positioned on the user's body in the most appropriate location for the sensors contained in the device. For example, a wearable sensor device containing an accelerometer or other motion sensor can be placed on the user's arm, leg, shoulder, back, head, etc. to best identify when the user moves. Similarly, a wearable sensor device containing a pulse oximeter may be positioned on the user's finger to best provide a reading of the user's hemoglobin saturation. Also, a wearable sensor device containing a heart rate monitor can be positioned on the user's chest to best sense heart beats.

In some embodiments, mobile device 101 can include an application for receiving the sensor data generated by any of the sensors in the one or more wearable sensor devices worn by a user. The application can also control one or more sensors on mobile device 101 to obtain additional sensor data representing environmental conditions around mobile device 101. For example, mobile device 101 can include a microphone for detecting audible sounds that may occur while the user is sleeping. Similarly, mobile device 101 can include a light sensor (e.g. the light sensor used to control the screen brightness of a smart phone) for detecting the presence of light while the user is sleeping. Also, mobile device 101 can include a camera for capturing an image or series of images of the user while the user is sleeping.

The application on mobile device 101 can receive sensor data from both the one or more sensors in the wearable sensor device or devices worn by the user and the one or more sensors within mobile device 101, and correlate the two types of sensor data to provide an indication of why a user performs some action during sleep.

For example, when a user moves his arm while sleeping, an accelerometer within wearable sensor device 102 attached to the user's arm can generate sensor data representing the movement of the user's arm. This sensor data can be transmitted by wearable sensor device 102 to mobile device 101. Additionally, a microphone within mobile device 101 can detect a sound and generate sensor data representing the occurrence of the sound.

The application on mobile device 101 can process the sensor data representing the movement of the user's arm and the sensor data representing the occurrence of the sound to identify that the sound occurred shortly before the movement of the user's arm. The proximity of the occurrence of the sound to the movement of the user's arm can indicate that the sound likely caused the user to move his arm. The application on mobile device 101 can then store a correlation between the sound and the movement to indicate that the user likely moved in response to the sound.

In this way, a better indication of the user's sleep patterns can be provided. For example, mobile device 101 can track such correlations that may occur during the user's sleep and generate an analysis that indicates how much of the user's movement during sleep was likely caused by external or environmental factors such as sound or light. By having such an analysis, the user can know that any issues with his sleep patterns are not likely due to any internal problems the user may have, but are more likely a result of the external occurrences of sound, light, or other environmental occurrence detectable by a sensor on a mobile device.

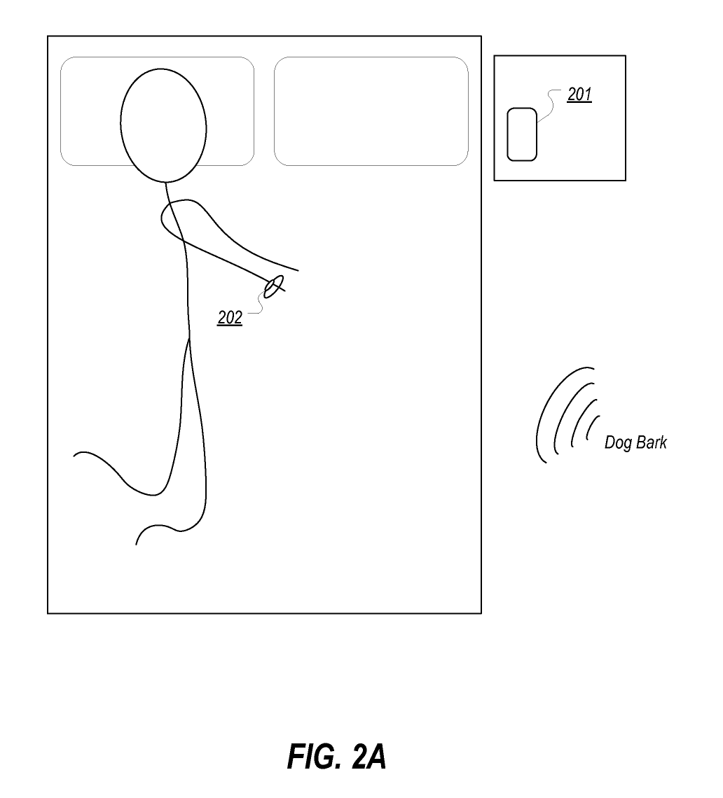

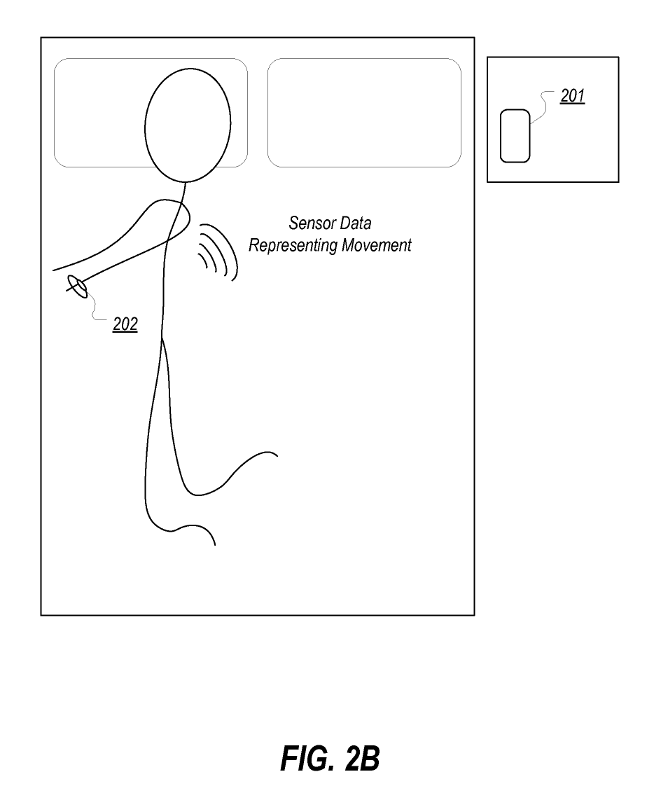

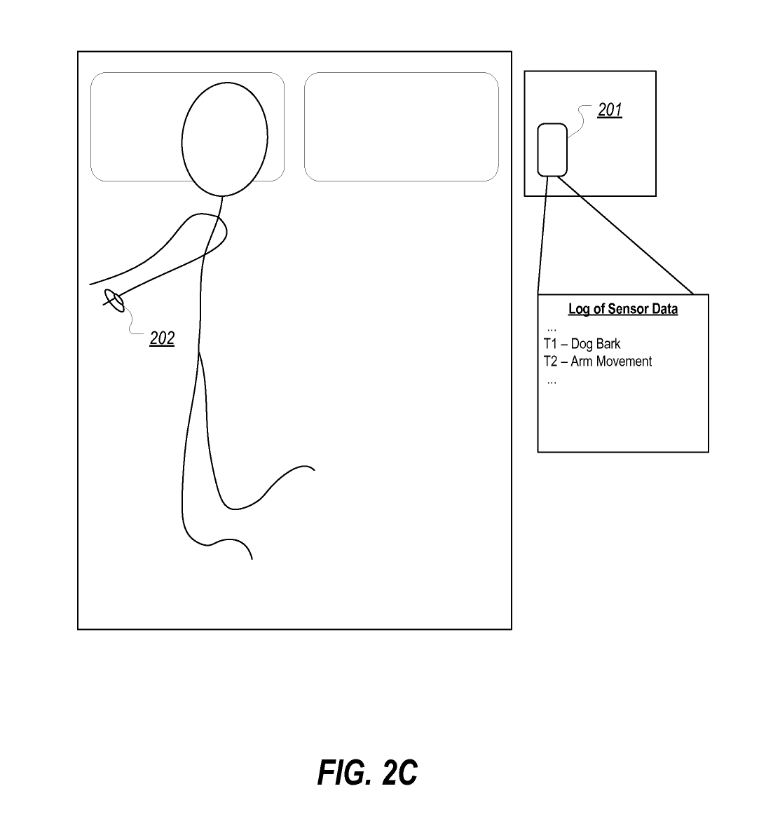

FIGS. 2A-2C represent how motion data generated by a wearable sensor device 202 can be correlated with audible data generated by a microphone of mobile device 201. FIG. 2A illustrates that, while the user is sleeping, a dog bark is audible within the user's bedroom at a first time. A microphone in mobile device 201 can be used to sense the dog bark and generate sensor data representing the occurrence of the dog bark at the first time. Prior to the dog bark, the user is sleeping motionless with his arms to the right.

FIG. 2B illustrates that at a subsequent time after the dog bark was audible within the user's bedroom, the user has moved so that his arms are to the left. This movement of the user's arm can be sensed by an accelerometer, gyroscope, or other motion sensor within wearable sensor device 202 causing sensor data representing the movement to be transmitted to mobile device 201.

FIG. 2C illustrates that mobile device 201 can store a log of sensor data received from one or more sensors of mobile device 201 and wearable sensor device 202. This log includes an indication that at time, t1, the dog bark occurred, and at time, t2, the user moved his arm. Mobile device 201 can analyze these two entries in the log to determine whether a correlation exists. In some embodiments, this analysis includes determining if the duration of time between the dog bark at t1 and the movement at t2 indicates that the movement was likely a result of the dog bark. For example, if the duration between t1 and t2 is below some threshold, a correlation between the dog bark and movement can be created.

In some cases, a correlation can be given a strength. For example, if the movement occurs immediately after or during the dog bark, a strong correlation can be indicated whereas a weaker correlation can be indicated as the duration between the dog bark and the movement increases. Similarly, the strength of the correlation can be based on how loud the dog bark was. For example, if the dog bark is loud, the strength of the correlation can be higher than when the dog bark is soft.

Similar strengths of the correlation can be created when the sensor data obtained from a sensor of mobile device 201 is from a light or other sensor. For example, the occurrence of a brighter light can result in a higher correlation strength than the occurrence of a dimmer light.

In addition to creating correlations between a user's movements and environmental occurrences, some embodiments of the present invention can also create correlations between the user's physiological parameters and an environmental occurrence. For example, a heart rate sensor within wearable sensor device 202 (or another wearable sensor device the user is wearing) can transmit the user's heart rate to mobile device 201. When there is an environmental occurrence such as a sound or a light, the heart rate of the user at the time of the environment occurrence can be correlated with the environmental occurrence.

For example, if mobile device 201 identifies that the user's heart rate spikes at time t2 and a loud sound was audible at time t1, mobile device 201 can determine whether the duration between t1 and t2 indicates that the spike in the heart rate was likely due to the loud sound and create a correlation accordingly.

Because the present invention allows the tracking and correlation of sensor data from both wearable sensor devices and mobile devices, the information that can be generated to represent the user's sleep patterns and activities can provide a more accurate indication of how the user is sleeping and why the user is performing certain actions during sleep. Without such correlations, the user is only informed of when the user moved but is not provided with any indication of why the user moved. This can cause the user to assume there is something wrong with his sleep patterns when in fact the problem is due only to external factors. Accordingly, the present invention allows wearable sensor devices to be used to provide much more useful information regarding the sleep of a user.

Correlating Sensor Data Obtained from a Wearable Sensor Device Worn During an Activity with Sensor Data Obtained from Sensors of a Mobile Device

The techniques described above for correlating a user's actions during sleep with environmental occurrences can also be used to correlate a user's actions during an activity with sensor data generated by sensors of a mobile device. For example, one or more sensors of mobile device 101 can be used to generate sensor data during a user's activity which is correlated with sensor data generated by one or more wearable sensor devices worn by the user during the activity.

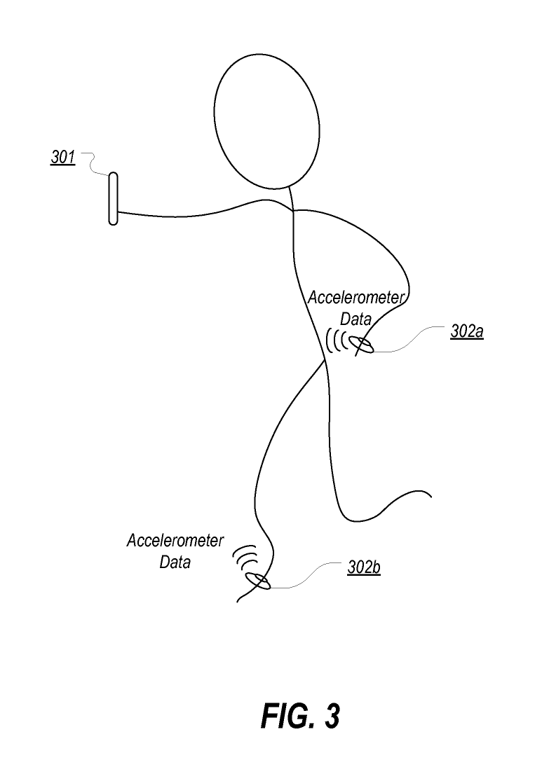

FIG. 3 illustrates an example of one type of correlation that can be performed during a user's activity which in this case is running. As shown, a user is wearing a wearable sensor device 302a on his wrist and a wearable sensor device 302b on his foot. Wearable sensor devices 302a and 302b are shown as including accelerometers for transmitting motion data to mobile device 301. Although two wearable sensor devices are shown, one or more wearable sensor devices that each contains any number or type of sensor can also be used.

FIG. 3 also shows that the user is holding mobile device 301 in order to take a picture of himself while running Accordingly, the camera of mobile device 301 can serve as a sensor for generating sensor data that can be correlated with sensor data received from wearable sensor device 302a and/or 302b.

In some embodiments, a correlation that can be made using a camera of mobile device 301 includes correlating the user's running form with accelerometer data. Such correlations can be used to identify where, in the user's stride, certain acceleration forces occur. Of course, similar correlations can be made when the user is performing another activity such as biking, swimming, yoga, golf, etc.

For example, a user can use mobile device 301 and one or more wearable sensor devices to create correlations between the position of his body during a golf swing and the acceleration forces identified in accelerometer data generated by the one or more wearable sensor devices. In this way, the user can more readily identify particular points in his swing that need to be improved.

Similar correlations can be made between images captured of the user during an activity and one or more physiological parameters represented in sensor data generated by the one or more wearable sensor devices. For example, a correlation can be created between a user's position and the user's heart rate or hemoglobin saturation.

In short, the present invention allows correlations to be made between sensor data generated by any of a number of sensors that can be provided on a mobile device and sensor data generated by one or more wearable sensor devices. In this way, the mobile device that receives sensor data from the wearable sensor devices can also receive sensor data from sensors contained in the mobile device to enable the generation of more useful information.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. A method, performed by a mobile device, for correlating sensor data received from a wearable sensor device with sensor data received from one or more sensors within the mobile device, the method comprising:
receiving first sensor data from one or more sensors of the mobile device, the first sensor data representing an environmental occurrence while a user is sleeping;
storing the first sensor data with an indication of a first time at which the first sensor data was generated;
receiving second sensor data from one or more wearable sensor devices that are worn by the user while the user is sleeping, the second sensor data being generated by one or more sensors in the one or more wearable sensor devices, the second sensor data representing a movement the user made while sleeping;
storing the second sensor data with an indication of a second time at which the second sensor data was generated;
analyzing the first and second sensor data including determining that the duration of time between the first time and the second time is below a specified threshold; and
creating a correlation between the environment occurrence and the movement.

2. The method of claim 1, wherein the environmental occurrence is one of a sound or light.

3. The method of claim 1, wherein the correlation includes a strength.

4. The method of claim 3, wherein the strength of the correlation is based on one or more of:
an intensity of the environmental occurrence; or
the duration of time between the first time and the second time.

5. The method of claim 1, wherein the one or more sensors of the mobile device comprise a microphone.

6. The method of claim 1, wherein the one or more sensors in the one or more wearable sensor devices comprise one or more accelerometers.

7. A method, performed by a mobile device, for correlating sensor data received from a wearable sensor device with sensor data received from one or more sensors within the mobile device, the method comprising:
receiving first sensor data from one or more sensors of the mobile device;
storing the first sensor data with an indication of a first time at which the first sensor data was generated;
receiving second sensor data from one or more wearable sensor devices that are worn by the user while the user is sleeping, the second sensor data being generated by one or more sensors in the one or more wearable sensor devices, the second sensor data representing a change in a physiological parameter of the user while the user is sleeping;
storing the second sensor data with an indication of a second time at which the second sensor data was generated;
analyzing the first and second sensor data including determining that the duration of time between the first time and the second time is below a specified threshold; and
creating a correlation between the environment occurrence and the change in the physiological parameter.

8. The method of claim 7, wherein the physiological parameter is the user's heart rate.

9. The method of claim 7, wherein the physiological parameter is the user's hemoglobin saturation.

10. The method of claim 7, wherein the correlation includes a strength.

11. The method of claim 10, wherein the strength of the correlation is based on one or more of:
an intensity of the environmental occurrence; or
the duration of time between the first time and the second time.

12. The method of claim 7, wherein the one or more sensors of the mobile device comprise a microphone.

13. The method of claim 7, wherein the one or more sensors in the one or more wearable sensor devices comprise one or more accelerometers.

14. One or more computer storage media storing computer executable instructions which when executed perform a method for correlating sensor data received from a wearable sensor device with sensor data received from one or more sensors within a mobile device, the method comprising:
receiving first sensor data from one or more sensors of the mobile device;
storing the first sensor data with an indication of a first time at which the first sensor data was generated;
receiving second sensor data from one or more wearable sensor devices that are worn by the user, the second sensor data being generated by one or more sensors in the one or more wearable sensor devices;
storing the second sensor data with an indication of a second time at which the second sensor data was generated;
analyzing the first and second sensor data including determining that the duration of time between the first time and the second time is below a specified threshold; and
creating a correlation between first sensor data and the second sensor data based on the duration of time being below the specified threshold.

15. The computer storage media of claim 14, wherein the first sensor data represents an audible occurrence, and the second sensor data represents motion.

16. The computer storage media of claim 14, wherein the first sensor data represents a visible occurrence, and the second sensor data represents motion.

17. The computer storage media of claim 14, wherein the second sensor data is generated while the user is sleeping.

18. The computer storage media of claim 14, wherein the second sensor data is generated while the user is exercising.

19. The computer storage media of claim 14, wherein the second sensor data represents motion, and the correlation indicates that the motion likely occurred as a result of an occurrence represented by the first sensor data.

20. The computer storage media of claim 14, wherein the method further comprises:
displaying, on the mobile device, an indication of the correlation.
